SEQUENCE LISTING (7 sequences)

SEQ ID NO: 1 - DNA, 1323 bp, Escherichia coli
atgcaagcctattttgaccagctcgatcgcgttcgttatgaaggctcaaaatcctcaaacccgttagcattccgtcactacaatcccgacgaactggtgttgggtaagcgtatggaagagcacttgcgttttgccgcctgctactggcacaccttctgctggaacggggcggatatgtttggtgtgggggcgtttaatcgtccgtggcagcagcctggtgaggcactggcgttggcgaagcgtaaagcagatgtcgcatttgagtttttccacaagttacatgtgccattttattgcttccacgatgtggatgtttcccctgagggcgcgtcgttaaaagagtacatcaataattttgcgcaaatggttgatgtcctggcaggcaagcaagaagagagcggcgtgaagctgctgtggggaacggccaactgctttacaaaccctcgctacggcgcgggtgcggcgacgaacccagatcctgaagtcttcagctgggcggcaacgcaagttgttacagcgatggaagcaacccataaattgggcggtgaaaactatgtcctgtggggcggtcgtgaaggttacgaaacgctgttaaataccgacttgcgtcaggagcgtgaacaactgggccgctttatgcagatggtggttgagcataaacataaaatcggtttccagggcacgttgcttatcgaaccgaaaccgcaagaaccgaccaaacatcaatatgattacgatgccgcgacggtctatggcttcctgaaacagtttggtctggaaaaagagattaaactgaacattgaagctaaccacgcgacgctggcaggtcactctttccatcatgaaatagccaccgccattgcgcttggcctgttcggttctgtcgacgccaaccgtggcgatgcgcaactgggctgggacaccgaccagttcccgaacagtgtggaagagaatgcgctggtgatgtatgaaattctcaaagcaggcggtttcaccaccggtggtctgaacttcgatgccaaagtacgtcgtcaaagtactgataaatatgatctgttttacggtcatatcggcgcgatggatacgatggcactggcgctgaaaattgcagcgcgcatgattgaagatggcgagctggataaacgcatcgcgcagcgttattccggctggaatagcgaattgggccagcaaatcctgaaaggccaaatgtcactggcagatttagccaaatatgctcaggaacatcatttgtctccggtgcatcagagtggtcgccaggaacaactggaaaatctggtaaaccattatctgttcgacaaataa

SEQ ID NO: 2 - DNA, 1440 bp, Escherichia coli
atgtcagtacccgttcaacatcctatgtatatcgatggacagtttgttacctggcgtggagacgcatggattgatgtggtaaaccctgctacagaggctgtcatttcccgcatacccgatggtcaggccgaggatgcccgtaaggcaatcgatgcagcagaacgtgcacaaccagaatgggaagcgttgcctgctattgaacgcgccagttggttgcgcaaaatctccgccgggatccgcgaacgcgccagtgaaatcagtgcgctgattgttgaagaagggggcaagatccagcagctggctgaagtcgaagtggcttttactgccgactatatcgattacatggcggagtgggcacggcgttacgagggcgagattattcaaagcgatcgtccaggagaaaatattcttttgtttaaacgtgcgcttggtgtgactaccggcattctgccgtggaacttcccgttcttcctcattgcccgcaaaatggctcccgctcttttgaccggtaataccatcgtcattaaacctagtgaatttacgccaaacaatgcgattgcattcgccaaaatcgtcgatgaaataggccttccgcgcggcgtgtttaaccttgtactggggcgtggtgaaaccgttgggcaagaactggcgggtaacccaaaggtcgcaatggtcagtatgacaggcagcgtctctgcaggtgagaagatcatggcgactgcggcgaaaaacatcaccaaagtgtgtctggaattggggggtaaagcaccagctatcgtaatggacgatgccgatcttgaactggcagtcaaagccatcgttgattcacgcgtcattaatagtgggcaagtgtgtaactgtgcagaacgtgtttatgtacagaaaggcatttatgatcagttcgtcaatcggctgggtgaagcgatgcaggcggttcaatttggtaaccccgctgaacgcaacgacattgcgatggggccgttgattaacgccgcggcgctggaaagggtcgagcaaaaagtggcgcgcgcagtagaagaaggggcgagagtggcgttcggtggcaaagcggtagaggggaaaggatattattatccgccgacattgctgctggatgttcgccaggaaatgtcgattatgcatgaggaaacctttggcccggtgctgccagttgtcgcatttgacacgctggaagatgctatctcaatggctaatgacagtgattacggcctgacctcatcaatctatacccaaaatctgaacgtcgcgatgaaagccattaaagggctgaagtttggtgaaacttacatcaaccgtgaaaacttcgaagctatgcaaggcttccacgccggatggcgtaaatccggtattggcggcgcagatggtaaacatggcttgcatgaatatctgcagacccaggtggtttatttacagtcttaa

SEQ ID NO: 3 - DNA, 747 bp, Caulobacter sp.
atgtcctcagccatctatcccagcctgaagggcaagcgcgtcgtcatcaccggcggcggctcgggcatcggggccggcctcaccgccggcttcgcccgtcagggcgcggaggtgatcttcctcgacatcgccgacgaggactccagggctcttgaggccgagctggccggctcgccgatcccgccggtctacaagcgctgcgacctgatgaacctcgaggcgatcaaggcggtcttcgccgagatcggcgacgtcgacgtgctggtcaacaacgccggcaatgacgaccgccacaagctggccgacgtgaccggcgcctattgggacgagcggatcaacgtcaacctgcgccacatgctgttctgcacccaggccgtcgcgccgggcatgaagaagcgtggcggcggggcggtgatcaacttcggttcgatcagctggcacctggggcttgaggacctcgtcctctacgaaaccgccaaggccggcatcgaaggcatgacccgcgcgctggcccgggagctgggtcccgacgacatccgcgtcacctgcgtggtgccgggcaacgtcaagaccaagcgccaggagaagtggtacacgcccgaaggcgaggcccagatcgtggcggcccaatgcctgaagggccgcatcgtcccggagaacgtcgccgcgctggtgctgttcctggcctcggatgacgcgtcgctctgcaccggccacgaatactggatcgacgccggctggcgttga

SEQ ID NO: 4 - DNA, 61 bp, Artificial Sequence
  XylK Forward primer
tcgtgaaggttacgaaacgctgttaaataccgacttgcgtcatatgaatatcctccttagt

SEQ ID NO: 5 - DNA, 61 bp, Artificial Sequence
  XylK Reverse primer
cggctcatgccgctgaacccatagcaatttaggcgcagtagtgtaggctggagctgcttcg

SEQ ID NO: 6 - DNA, 19 bp, Artificial Sequence
  XylA Forward primer
cggcaacgcaagttgttac

SEQ ID NO: 7 - DNA, 19 bp, Artificial Sequence
  XylA Reverse primer
cgtcagacatatcgctggc

(19) United States
(12) Patent Application Publication
(10) Pub. No.: US 2015/0147794 A1
(43) Pub. Date: May 28, 2015

(54) ETHANE-1,2-DIOL PRODUCING MICROORGANISM AND A METHOD FOR PRODUCING ETHANE-1,2-DIOL FROM D-XYLOSE USING THE SAME

(71) Applicant: MYONGJI UNIVERSITY INDUSTRY AND ACADEMIA COOPERATION FOUNDATION, Yongin-si, Gyeonggi-do (KR)

(72) Inventors: Wook-Jin Chung, Seongnam-si (KR); Huaiwei Liu, Yongin-si (KR)

(21) Appl. No.: 14/376,800

(22) PCT Filed: Feb. 5, 2013
(86) PCT No.: PCT/KR2013/000917

§ 371 Date: Aug. 5, 2014

(30) Foreign Application Priority Data
Feb. 6, 2012 (KR) 10-2012-0011913

(51) Int. Cl. C12P 7/18; C12N 9/04; C12N 15/70

(52) U.S. Cl. CPC C12P 7/18; C12N 15/70; C12N 9/0006; C12Y 101/01175
USPC 435/158; 435/252.33; 435/471

(57) ABSTRACT

Disclosed herein is a microorganism capable of producing ethane-1,2-diol from D-xylose, and a method for producing ethane-1,2-diol using the same. More specifically, the present invention relates to an engineered Escherichia coli (E. coli) prepared by knocking out a D-xylose isomerase gene and/or an aldehyde dehydrogenase gene within the genomic DNA of E. coli and transforming an expression vector including a D-xylose dehydrogenase gene into the E. coli, and an efficient method for producing ethane-1,2-diol from D-xylose using the engineered E. coli.

### BACKGROUND OF THE INVENTION

1. Field of the Invention

The present invention relates generally to a method for the biosynthesis of ethane-1,2-diol and, more particularly, to an ethane-1,2-diol producing microorganism and a method for producing ethane-1,2-diol from D-xylose using the same.

2. Description of the Related Art

As well known in the art, ethane-1,2-diol (ethylene glycol; EG) is an important platform chemical used as a polymer precursor as well as an antifreeze and a coolant (Non-patent Documents 1 & 2). There has been a growing global demand on ethane-1,2-diol, for example, the global demand was 17.8 million tons in 2010 and is expected to reach about 23.6 million tons in 2014 (Non-patent Document 3).

Since ethane-1,2-diol has been commercially produced from ethylene, a major product in petrochemical industry (Non-patent Document 4), its production largely depends on fossil fuels and is limited as such. Due to the global demand on the technical development for producing chemicals and materials from renewable biomass rather than from fossil resources, there have been reports recently on green chemistry technologies capable of producing ethane-1,2-diol from biomass (Non-patent Document 5). Examples of the technologies may include hydrogenolysis of xylitol using a Ru/C catalyst under 4.0 MPa of H₂ gas pressure and at 473 K of reaction temperature (Non-patent Document 6), and a technology performing a rapid pyrolysis of lignocellulosic biomass followed by a combination of an hydrogenation process and zeolite catalysis (Non-patent Document 7). These technologies share the common feature that various products are formed under high pressure and temperature conditions through a complicated downstream ethane-1,2-diol separation process. However, there has been no report on the technology for ethane-1,2-diol biosynthesis.

Accordingly, the inventors of the present invention, after numerous efforts for the development of ethane-1,2-diol biosynthesis, designed a biosynthesis route for ethane-1,2-diol production from D-xylose, second most-abundant sugar in lignocellulosic feedstocks, and by applying the biosynthesis route to E. coli, prepared an engineered E. coli, which enables a large-scale ethane-1,2-diol production using D-xylose while considerably lowering the amount of byproducts, and confirmed that ethane-1,2-diol can be efficiently produced from D-xylose using the engineered E. coli, thereby completing the present invention.

### PRIOR ART DOCUMENTS

1. Aggarwal, S. L.; Sweeting, O. J. Chem. Rev. 1957, 57, 665-742.
2. Baudot, A.; Odagescu, V. Cryobiology. 2004, 48, 283-94.
3. Himfr, T. http://www.articlesurge.com. 2011.
4. Wishart, R. S. Science. 1978, 10, 614-618.
5. Lee, J. W.; Kim, T. Y.; Jang, Y. S.; Choi, S.; Lee, S. Y. Trends. Biotechnol. 2011, 29, 370-378.
6. Sun, J., Liu, H. Green Chem. 2011, 13, 135-142.
7. Vispute, T. P.; Zhang, H.; Sanna, A.; Xiao, R.; Huber, G. W. Science. 2010, 330, 1222-1227.
8. Niu, W.; Molefe, M. N.; Frost, J. W. J. Am. Chem. Soc. 2003, 125, 12998-12999.
9. Yim, H., et al. Nat. Chem. Biol. 2011, 7, 445-452.
10. Liu, H.; Valdehuesa, K. N.; Nisola, G. M.; Ramos, K. R.; Chung, W. J. Bioresour. Technol. 2011, doi: 10.1016/j.biortech.2011.08.065.
11. Frost, J. W. MEWG: Interagency Conference on Metabolic Engineering. 2008, North Bethesda, Md.
12. Jarboe, L. R. Appl. Microbiol. Biotechnol. 2011, 89, 249-57.
13. Mavrovouniotis, M. L. Biotechnol. Bioeng. 1999, 36, 1070-1082.
14. Baba, T.; Ara, T.; Hasegawa, M.; Takai, Y.; Okumura, Y.; Baba, M.; Datsenko, K. A.; Tomita, M.; Wanner, B. L.; Mori, H. Mol. Syst. Biol. 2006, 2, 2006.0008.
15. Datsenko, K. A.; Wanner, B. L. Proc. Natl. Acad. Sci. 2000, 97, 6640-6645.
16. Liu, H.; Valdehuesa, K. N.; Nisola, G. M.; Ramos, K. R.; Chung, W. J. Bioresour. Technol. 2011, doi: 10.1016/j.biortech.2011.08.065.
17. Mavrovouniotis, M. L. Biotechnol. Bioeng. 1999, 36, 1070-1082.

### SUMMARY OF THE INVENTION

Accordingly, the present invention has been made keeping in mind the above problems occurring in the prior art, and an object of the present invention is to provide an efficient method of producing ethane-1,2-diol from D-xylose, the second most-abundant sugar in lignocellulosic feedstocks.

In order to accomplish the above object, the present invention provides an engineered E. coli capable of producing ethane-1,2-diol from D-xylose, which can perform the biosynthesis route for ethane-1,2-diol production according to the present invention.

Additionally, the present invention also provides a method for ethane-1,2-diol production including culturing the engineered E. coli in a medium containing D-xylose.

Additionally, the present invention also provides a method for preparing an engineered E. coli including disruption and insertion of a gene so that the engineered E. coli can perform the biosynthesis route for ethane-1,2-diol production according to the present invention.

### ADVANTAGEOUS EFFECTS OF THE INVENTION

The present invention provides a method for an efficient large-scale ethane-1,2-diol production with high purity and high yield but with an extremely low level of byproducts; achieved by designing a biosynthesis route for ethane-1,2-diol production from D-xylose, and confirmed by applying the biosynthesis route to E. coli using D-xylose as a substrate. In particular, the present invention, being the first pioneer invention regarding ethane-1,2-diol biosynthesis, provides a guideline for future biological production of ethane-1,2-diol.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a schematic diagram showing the chemical synthesis of ethylene glycol (*reaction condition: 473 K, 4.0 MPa H₂, Ru/C);

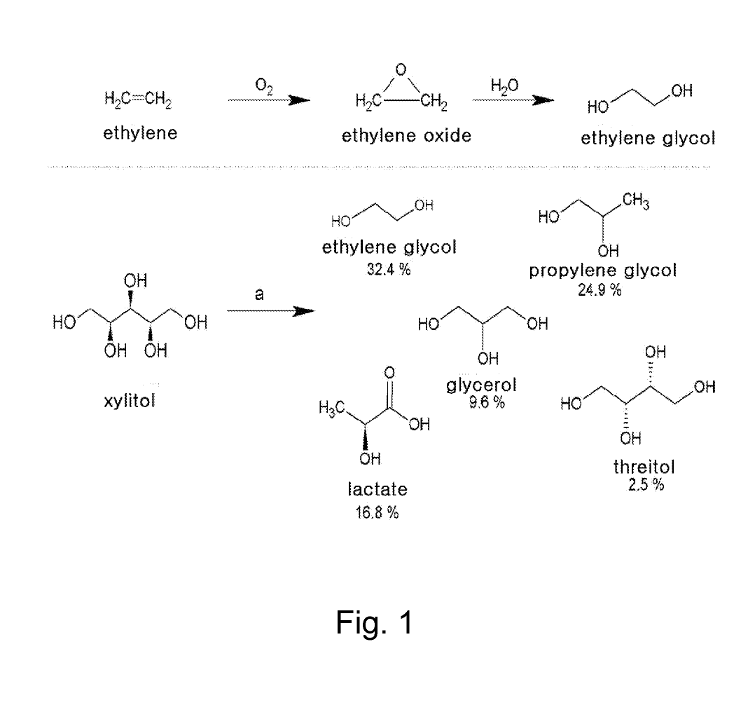

FIG. 2 is a schematic diagram showing a biosynthesis route for producing ethylene glycol in E. coli^{a}: ^{a}enzyme:

(1) D-xylose dehydrogenase (Caulobacter crescentus (C. crescentus));

(2) D-xylonic acid dehydratase (E. coli);

(3) 2-dehydro-3-deoxy-D-pentonate aldolase (E. coli);

(4) dehydrogenase (E. coli);

(b1) D-xylose isomerase (E. coli); and

(b2) aldehyde dehydrogenase (E. coli);

FIG. 3 is a schematic diagram showing a biosynthesis route for producing ethylene glycol from D-xylose using E. coli;

FIG. 4 is a schematic diagram showing a map of pET28a-cxylB vector;

FIG. 5 is a graph showing the result of high performance liquid chromatography (HPLC) analysis on biosynthesized ethane-1,2-glycol in E. coli, in which the sample was taken from the fermentation product of E. coli W3110ΔylA::Cm^{r}(DE3)/pET28a-cxylB after 48 hours of fermentation;

FIG. 6 is a graph showing the result of gas chromatography (GC) analysis on biosynthesized ethane-1,2-glycol in E. coli, in which the sample was taken from the fermentation product of E. coli W3110ΔxylA::Cm^{r}(DE3)/pET28a-cxylB after 48 hours of fermentation, in which 1,3-propanediol was used as an internal standard (IS);

FIG. 7 is a graph showing the result of gas chromotography-mass spectrometry (GC-MS) on biosynthesized ethane-1,2-glycol in E. coli, in which the sample was taken from the fermentation product of E. coli W3110ΔxylA::Cm^{r}(DE3)/pET28a-cxylB after 48 hours of fermentation, in which 1,3-propanediol was used as an IS;

FIG. 8 is a graph showing a time course of ethane-1,2-glycol in E. coli W3110ΔxylA::Cm^{r}(DE3)/pET28a-cxylB;

FIG. 9 is a graph showing a time course of ethane-1,2-glycol in E. coli BW25113ΔaldAΔxylA::Cm^{r}(DE3)/pET28a-cxylB; and

FIG. 109 is a schematic diagram showing the two biosynthetic routes for converting pyruvate into ethane-1,2-glycol^{a}: ^{a}enzyme:

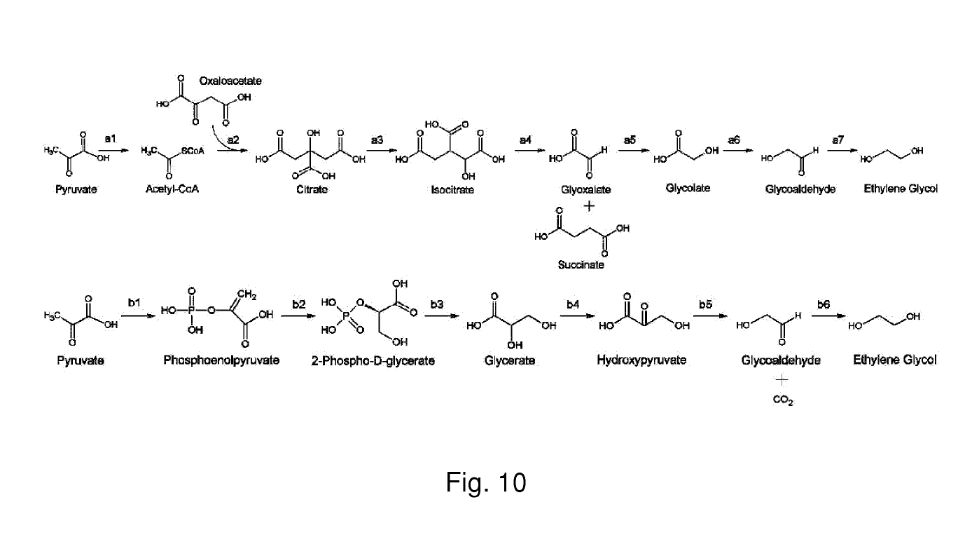

(a1) pyruvate dehydrogenase (E. coli);

(a2) citrate synthase (E. coli);

(a3) citrate hydrolyase (Citrate hydrolyase) (E. coli);

(a4) isocitrate lyase (E. coli);

(a5) glycolate oxidase (E. coli);

(a6), (a7) and (b6) aldehyde dehydrogenase (E. coli);

(b1) phosphoenolpyruvate synthase (E. coli);

(b2) enolase (E. coli);

(b3) 2-phosphoglycerate phosphatase (Veillonella alcalescens);

(b4) hydroxypyruvate reductase (E. coli); and

(b5) wide-range-substrate decarboxylase (Pseudomonas putida; Lactococcus lactis).

### DESCRIPTION OF THE PREFERRED EMBODIMENTS

The present invention will be described in detail herein below.

In an embodiment of the present invention, there is provided a method for preparing an engineered E. coli capable of producing ethane-1,2-diol from D-xylose by knocking out D-xylose isomerase gene, xylA, within the genomic DNA of E. coli followed by transforming an expression vector including D-xylose dehydrogenase gene, cxylB, into the xylA-knockout E. coli strain.

Preferably, the engineered E. coli is the strain deposited under the Deposition No. KCTC 12100BP but is not limited thereto.

In another embodiment of the present invention, there is provided a method for preparing an engineered E. coli capable of producing ethane-1,2-diol from D-xylose by knocking out the aldehyde dehydrogenase gene, aldA, within the genomic DNA of the xylA-knockout E. coli strain followed by transforming an expression vector including D-xylose dehydrogenase gene, cxylB into the aldA- and xylA-knockout E. coli strain.

Preferably, the engineered E. coli is the strain deposited under the Deposition No. KCTC 12117BP but is not limited thereto.

In the engineered E. coli, D-xylose isomerase gene, xylA, preferably includes a nucleotide sequence described in SEQ ID NO. 1 but is not limited thereto.

In the engineered E. coli, aldehyde dehydrogenase gene, aldA, preferably includes a nucleotide sequence described in SEQ ID NO. 2 but is not limited thereto.

In the engineered E. coli, D-xylose dehydrogenase gene, cxylB, being derived from Caulobacter crescentus (C. crescentus), preferably includes a nucleotide sequence described in SEQ ID NO: 3 but is not limited thereto.

In the engineered E. coli, the expression vector is preferably pET28a vector but is not limited thereto, and any vector which can express a target gene inserted therein, may be used.

In the engineered E. coli, the E. coli strain is preferably E. coli W3110 or E. coli BW25113 but is not limited thereto, and any E. coli strain may be used.

The engineered E. coli may produce ethane-1,2-diol via a four-step biosynthesis route using D-xylose as a substrate as described below.

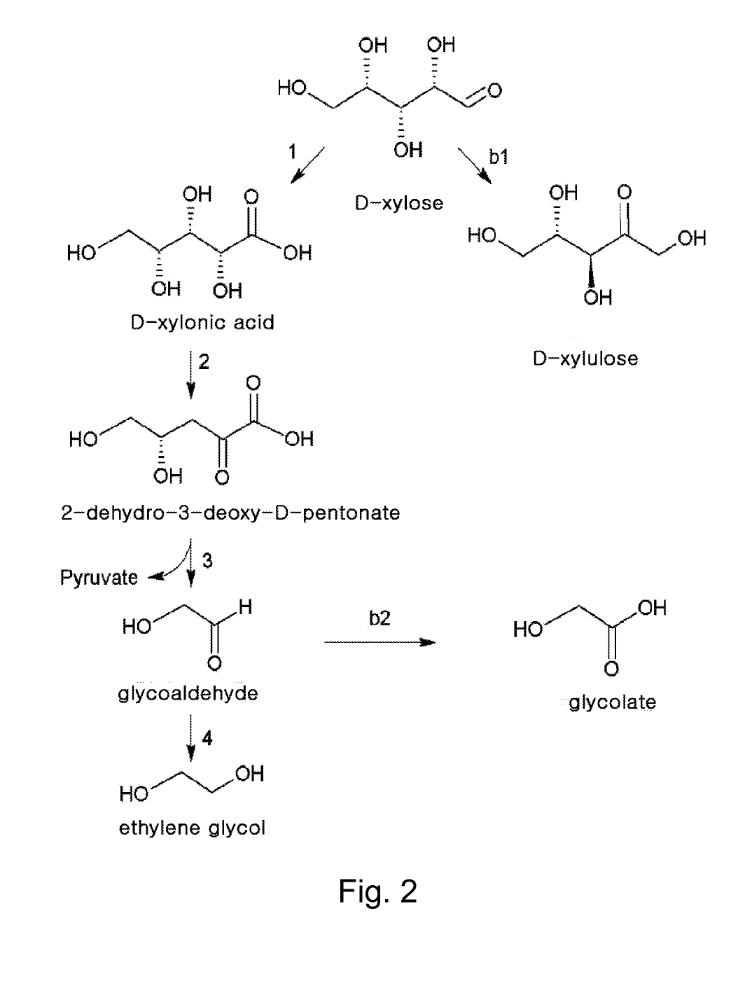

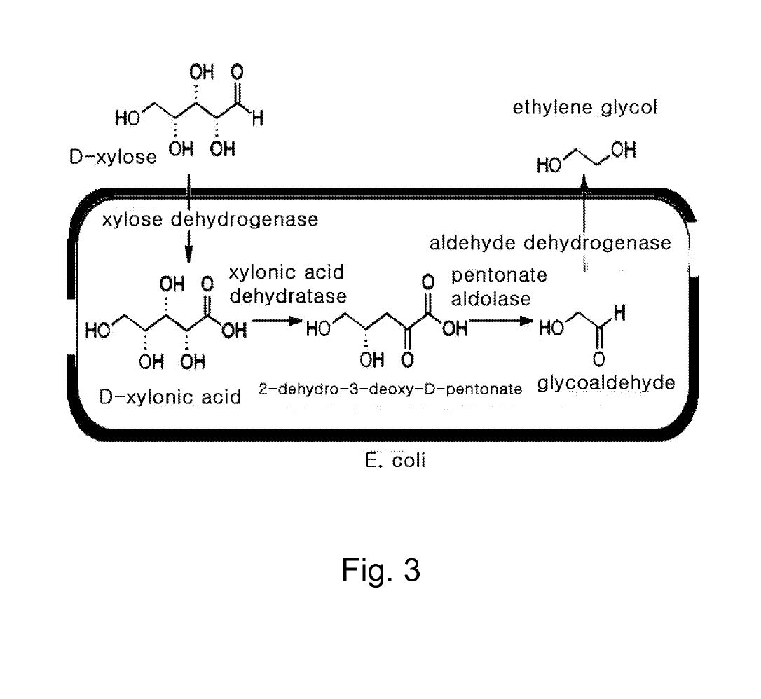

More specifically, the biosynthesis route may include a first step of converting D-xylose into D-xylonic acid by the catalytic activity of D-xylose dehydrogenase, a second step of converting the converted D-xylonic acid into 2-dehydro-3-deoxy-D-pentonate by the catalytic activity of D-xylonic acid dehydratase in E. coli, a third step of converting the converted 2-dehydro-3-deoxy-D-pentonate into glycoaldehyde by the catalytic activity of 2-dehydro-3-deoxy-D-pentonate aldolase in E. coli, and a fourth step of converting the converted glycoaldehyde into ethylene glycol by the catalytic activity of aldehyde dehydrogenase in E. coli (FIGS. 2 and 3).

In an embodiment of the present invention, a four-step biosynthesis route for ethane-1,2-diol production from D-xylose was designed, as shown in FIG. 2.

In an embodiment of the present invention, a thermodynamic analysis was performed in order to confirm the thermodynamic practicability of the designed biosynthesis route. The result showed that, among the four steps, the standard Gibbs free energy for the aldol decomposition reaction in the third step was low positive but it was negative for each of the other three steps, and also negative for the entire biosynthesis route. Accordingly, it was confirmed that the biosynthesis route is thermodynamically practicable.

In an embodiment of the present invention, pathway prediction system was analyzed via database, in order to predict potential reactions that may convert the intermediates generated in the biosynthesis route designed above to other byproducts. As a result, it was confirmed that the reactions of converting D-xylose into D-xylulose (step b1 in FIG. 2) and converting glycoaldehyde into glycolic acid (step b2 in FIG. 2) may be induced, respectively.

In an embodiment of the present invention, in order to perform the biosynthesis route designed above in E. coli, an engineered E. coli W3110ΔxylA::Cm^{r}(DE3)/pET28a-cxylB was prepared by a method including: preparing an E. coli W3110ΔylA::Cm^{r}(DE3), in which D-xylose isomerase gene xylA was disrupted within the genomic DNA of E. coli W3110, as a host cell; ligating C. crescentus-derived xylose dehydrogenase gene cxylB into pET28a vector to be regulated by T7 promoter; and transforming the recombinant plasmid pET28a-cxylB into the host cell.

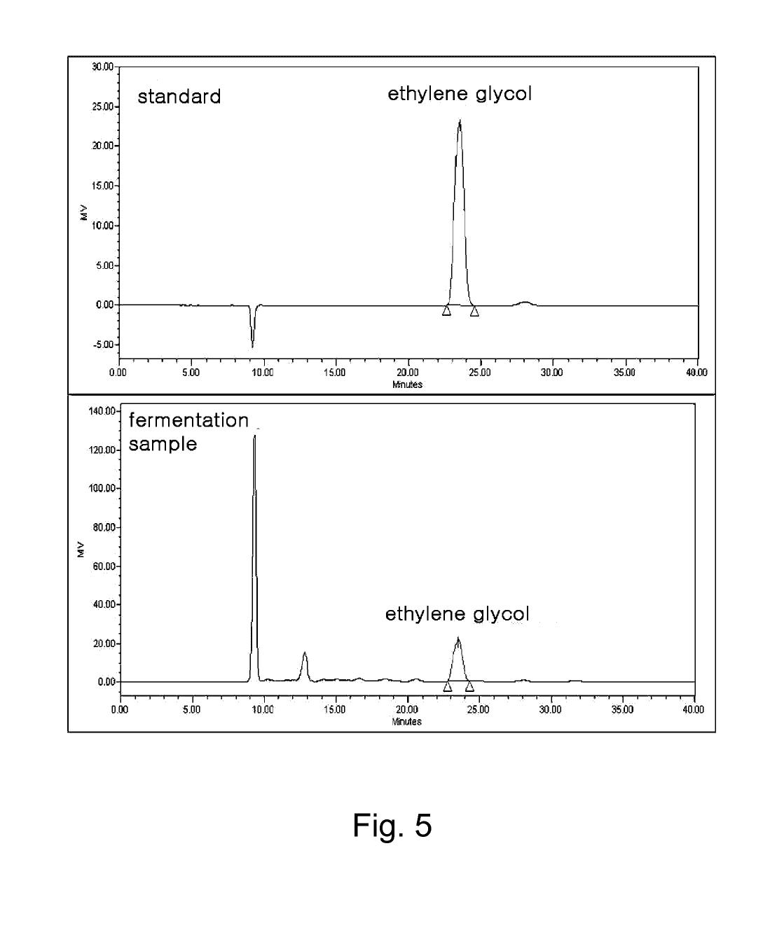

In an embodiment of the present invention, in order to confirm the ethane-1,2-diol production capacity of W3110ΔxylA::Cm^{r}(DE3)/pET28a-cxylB and E. coli BW25113ΔaldAΔxylA::Cm^{r}(DE3)/pET28a-cxylB, they were cultured in a medium containing D-xylose as a substrate, and the metabolic products contained in the culture were analyzed. According to the result, E. coli W3110ΔxylA::Cm^{r}(DE3)/pET28a-cxylB produced highly concentrated ethane-1,2-diol with high yield but byproducts at an extremely low level, and E. coli BW25113ΔaldAΔxylA::Cm^{r}(DE3)/pET28a-cxylB also produced highly concentrated ethane-1,2-diol with high yield, although not as high as those of E. coli W3110ΔxylA::Cm^{r}(DE3)/pET28a-cxylB (FIGS. 5-9).

In an embodiment of the present invention, in order to optimize the biosynthesis route designed as described above, an additionally designed biosynthesis route, in which metabolic engineering was further applied so as to improve yield and concentration of the product, was designed as shown in FIG. 10.

More specifically, it was confirmed the additionally designed biosynthesis route can increase the yield and the concentration of ethane-1,2-diol produced thereby, by converting pyruvate, which is produced during the conversion of 2-dehydro-3-deoxy-D-pentonate into glycoaldehyde by the catalytic activity of 2-dehydro-3-deoxy-D-pentonate aldolase in E. coli, in the third step of the biosynthesis route for ethane-1,2-diol production (FIG. 2), into ethane-1,2-diol (FIG. 10).

In an embodiment of the present invention, there is provided a method for producing ethane-1,2-diol from D-xylose, including:

1) biosynthesizing ethane-1,2-diol by culturing the two different strains of engineered E. coli of the present invention in a medium containing D-xylose; and

2) obtaining ethane-1,2-diol from the cultured medium.

In the production method described above, the engineered E. coli in step 1) is preferably E. coli W3110ΔxylA::Cm^{r}(DE3)/pET28a-cxylB or E. coli BW25113ΔaldAΔxylA::Cm^{r} (DE3)/pET28a-cxylB, but is not limited thereto.

In step 1) of the production method described above, the engineered E. coli is preferably cultured in a fermenter via batch fermentation, but is not limited thereto.

In the production method described above, the biosynthesis of ethane-1,2-diol in step 1) may include:

a) converting D-xylose into D-xylonic acid by D-xylose dehydrogenase;

b) converting D-xylonic acid into 2-dehydro-3-deoxy-D-pentonate by D-xylonic acid dehydratase;

c) converting 2-dehydro-3-deoxy-D-pentonate into glycoaldehyde by 2-dehydro-3-deoxy-D-pentonate aldolase; and

d) converting glycoaldehyde into ethane-1,2-diol by aldehyde dehydrogenase.

In particular, in the biosynthesis of ethane-1,2-diol in the engineered E. coli, in order to convert pyruvate, a byproduct produced in converting 2-dehydro-3-deoxy-D-pentonate into glycoaldehyde in step c), into ethane-1,2-diol, the method may further include:

e) converting pyruvate into acetyl-CoA by pyruvate dehydrogenase;

f) converting acetyl-CoA into citrate by citrate synthase by citrate synthase;

g) converting citrate into isocitrate by citrate hydrolyase;

h) converting isocitrate into glyoxalate and succinate by isocitrate lyase;

i) converting glyoxalate into glycolate by glycolate oxidase;

j) converting glycolate into glycoaldehyde by aldehyde dehydrogenase; and

k) converting glycoaldehyde into ethane-1,2-diol by aldehyde dehydrogenase.

Additionally, in the biosynthesis of ethane-1,2-diol in the engineered E. coli, in order to convert pyruvate, a byproduct produced in converting 2-dehydro-3-deoxy-D-pentonate into glycoaldehyde in step c), into ethane-1,2-diol, the method may further include:

l) converting pyruvate into phosphoenolpyruvate by phosphoenolpyruvate synthase;

m) converting phosphoenolpyruvate into 2-phospho-D-glycerate by enolase;

n) converting 2-phospho-D-glycerate into glycerate by 2-phosphoglycerate phosphatase;

o) converting glycerate into hydroxypyruvate by hydroxypyruvate reductase;

p) converting hydroxypyruvate into glycoaldehyde and CO₂ by decarboxylase; and

q) converting glycoaldehyde into ethane-1,2-diol by aldehyde dehydrogenase.

In an embodiment of the present invention, there is provided a method for producing ethane-1,2-diol from D-xylose, including:

1) knocking out D-xylose isomerase gene, xylA, from a given E. coli;

2) constructing an expression vector including xylose dehydrogenase gene, cxylB; and

3) transforming expression vector in step 2) into the resulting E. coli in step 1).

In an embodiment of the present invention, there is provided a method for producing ethane-1,2-diol from D-xylose, including:

1) knocking out aldehyde dehydrogenase gene, aldA, from a given E. coli;

2) knocking out D-xylose isomerase gene, xylA, from the resulting E. coli in step 1);

3) constructing an expression vector including xylose dehydrogenase gene, cxylB; and

4) transforming the expression vector constructed in step 3) into the resulting E. coli in step 2).

In the manufacturing method described above, D-xylose isomerase gene, xylA, should preferably include a nucleotide sequence described in SEQ ID NO: 1, but is not limited thereto.

In the manufacturing method described above, aldehyde dehydrogenase gene, aldA, should preferably include a nucleotide sequence described in SEQ ID NO: 2, but is not limited thereto.

In the manufacturing method described above, D-xylose dehydrogenase gene, cxylB, being derived from Caulobacter crescentus (C. crescentus), should preferably include a nucleotide sequence described in SEQ ID NO: 3, but is not limited thereto.

In the manufacturing method described above, the expression vector should preferably be pET28a vector, but is not limited thereto, and any vector enabling the expression of any inserted target gene in E. coli may be used.

In the manufacturing method described above, the E. coli should preferably be E. coli W3110 or E. coli BW25113, but is not limited thereto, and any E. coli may be used.

A better understanding of the present invention may be obtained through the following examples which are set forth to illustrate, but are not to be construed as the limit of the present invention. Accordingly, those skilled in the art will appreciate that various modifications, additions and substitutions are possible, without departing from the scope and spirit of the invention.

### Example 1

### Designing of a Biosynthesis Route for Ethane-1,2-Diol Production of the Present Invention

The inventors of the present invention designed a biosynthesis route for ethane-1,2-diol production from D-xylose in E. coli (FIG. 2), in which the first step of the biosynthesis route is to convert D-xylose into D-xylonic acid by the catalytic activity of D-xylose dehydrogenase; the second step is to convert D-xylonic acid into 2-dehydro-3-deoxy-D-pentonate by the catalytic activity of D-xylonic acid dehydratase in E. coli; the third step is to convert 2-dehydro-3-deoxy-D-pentonate into glycoaldehyde by the catalytic activity of 2-dehydro-3-deoxy-D-pentonate aldolase in E. coli; and the fourth step is to convert glycoaldehyde into ethane-1,2-diol by the catalytic activity of aldehyde dehydrogenase in E. coli.

More specifically, in the first step of the biosynthesis route of the present invention, D-xylose dehydrogenase was used to convert D-xylose into D-xylonic acid. Since D-xylose dehydrogenase in each microorganism has its own characteristics, a D-xylose dehydrogenase derived from Caulobacter crescentus was selected for the reaction described above. The selected D-xylose dehydrogenase prefers NAD⁺, a coenzyme, to NADP⁺ (Non-patent Document 10). NAD⁺ can be regenerated via various reactions in the cellular metabolic network, and thus the depletion of the coenzyme can be prevented in the first step. E. coli encodes D-xylonic acid dehydratase (YjhG and YagF) which can promote the second step of the reaction, and encodes two 2-dehydro-3-deoxy-D-pentonate aldolases (YjhH and YagE) which can promote the third step of the reaction (Non-patent Documents 10 & 11). Based on the enzyme profile described above, E. coli was selected as a host. Additionally, the broad-substrate-range of the aldehyde dehydrogenase YqhD can promote the final step of the biosynthesis route of the present invention (Non-patent Document 12). Considering other aldehyde dehydrogenases' versatility and diversity in E. coli, the intrinsic activity of dehydrogenase is suitable for performing the fourth step in E. coli.

### Example 2

### Thermodynamic Analysis of a Biosynthesis Route for Ethane-1,2-Diol Production of the Present Invention

A thermodynamic analysis was performed for the theoretical evaluation of the biosynthesis route for ethane-1,2-diol production of the present invention (FIG. 2) regarding its thermodynamic practicability. In order to calculate the standard Gibbs free energy change (ΔᵣG′°) for each reaction, a group contribution method was applied thereto (Non-patent Document 13). All ΔᵣG′° values relating to reaction schemes are shown in Table 1 below.

TABLE 1
ΔrG′° values in biosynthesis route for ethane-1,2-
diol production

| | | | ΔrG′° |
|---|---|---|---|
| Step | Reaction | Enzyme | (kcal/mol) |
| 1 | D-xylose + NAD⁺ + H₂O → D- | D-xylose | −14.1 |
| | xylonate + NADH + 2H⁺ | dehydrogenase | |
| 2 | D-xylonate → 2-keto-3- | D-xylonate | −8.6 |
| | deoxy-D-xylonate + H₂O | dehydratase | |
| 3 | 2-keto-3-deoxy-D-xylonate | 2-dehydro-3-deoxy-D- | 4.3 |
| | → glycoaldehyde + | pentonate aldolase | |
| | pyruvate | | |
| 4 | glycoaldehyde + NAD(P) H + | glycoaldehyde | −7.1 |
| | H+ → ethane-1,2-glycol + | dehydrogenase | |
| | NAD(P)⁺ | | |

The result showed that, among the four different reactions of the biosynthesis route (FIG. 2), the aldol decomposition reaction in step 3 had a small positive ΔᵣG′° value, indicating that it is an independent reaction not preferred thermodynamically. However, the result showed that the remaining three reactions had negative ΔᵣG′° values, and the total standard Gibbs free energy of the entire biosynthesis route was shown as negative. Accordingly, from the theoretical point of view, the biosynthesis route (FIG. 2) of the present invention is thermodynamically practicable.

### Example 3

### Confirmation of Predictability of Other Byproducts in the Biosynthesis Route for Ethane-1,2-Diol Production of the Present Invention

The predictability of a potential reaction capable of converting the intermediate products of the biosynthesis route (FIG. 2) for ethane-1,2-diol production into other byproducts was analyzed by a route prediction system of University of Minnesota Biocatalysis and Biodegradation Database (UM-BBD).

The result confirmed that the two reactions of b1 and b2 in the biosynthesis route for ethane-1,2-diol production of the present invention could occur in E. coli as shown in FIG. 2. Xylose isomerase (XI) and aldehyde dehydrogenase (AldA) were shown to exhibit their respective catalytic activities in the two reactions described above (EcoCYC).

### Example 4

### Performance of a Biosynthesis Route for Ethane-1,2-Diol Production of the Present Invention in E. coli

<4-1> Preparation of Strains

E. coli W3110 was purchased from American Type Culture Collection (ATCC; ATCC No. 27325), and E. coli BW25113ΔaldA::Kan^{r} was purchased from Keio collection of National BioResource Project (NBRP) (Non-patent Document 14).

The one-step gene inactivation strategy derived from the previous reports of Datsenko and Wanner were applied for the gene disruption and removal of resistant genes from the genomic DNA of E. coli (Non-patent Document 15). All the gene disruption primers and probing primers are shown in Table 2 below.

TABLE 2

| Primer | Sequence (5′-3′) | Function |
|---|---|---|
| XylK-F | TCGTGAAGGTTACGAAACGC | Amplify |
| | TGTTAAATACCGACTTGCGT | fragments of |
| | CATATGAATATCCTCCTTAG | disrupted |
| | T (SEQ ID NO: 4) | xylA gene |
| XylK-R | CGGCTCATGCCGCTGAACCC | |
| | ATAGCAATTTAGGCGCAGTA | |
| | GTGTAGGCTGGAGCTGCTTC | |
| | G (SEQ ID NO: 5) | |
| XylA-F | CGGCAACGCAAGTTGTTAC | Verify |
| | (SEQ ID NO: 6) | disruption |
| | | of xylA gene |
| XylA-R | CGTCAGACATATCGCTGGC | |
| | (SEQ ID NO: 7) | |

<4-2> Construction of Plasmids

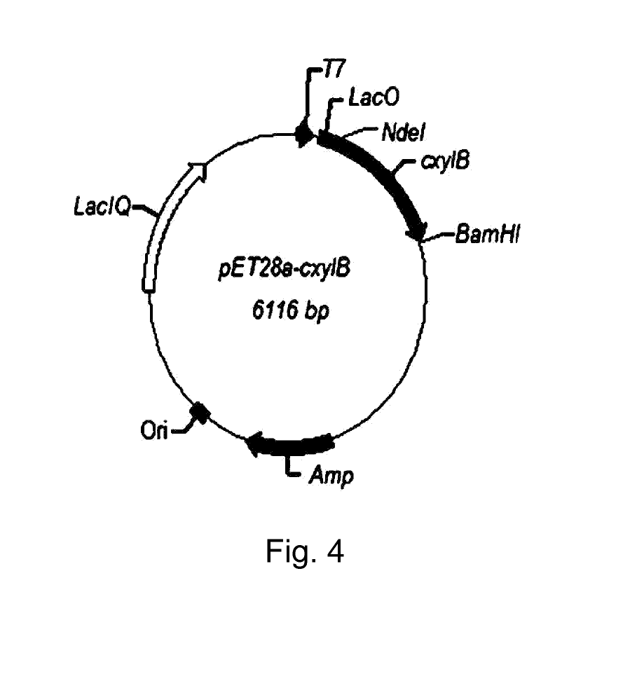

Plasmid pET28a-cxylB was constructed in advance (FIG. 4) (Non-patent Document 16). Plasmid pKD46 was used as a Red recombinase expression vector, pKD3 as a template plasmid for PCR amplification of disruption cassettes, and pCP20 as a plasmid for removal of resistant genes. The protocol used for gene disruption and removal was according to the OPENWETWARE (Http://openwetware.org).

<4-3> Preparation of E. coli W3110ΔxylA::Cm^{r} (DE3)/pET28a-cxylB

The xylA disruption cassette was amplified with a pair of disruption primers using pKD3 as a template. The amplified disruption cassette was applied to E. coli W3110 and thereby obtained E. coli W3110ΔxylA::Cm^{r}. E. coli W3110ΔxylA::Cm^{r} disrupted D-xylose isomerase gene xylA thereby preventing the conversion of D-xylose into D-xylulose.

Upon confirmation of genotypes and phenotypes of gene disruption, λDE3 prophage was inserted into E. coli W3110 ΔxylA::Cm^{r} using a λDE3 lysogenization kit (Novagen, USA), and finally obtained a construct of E. coli W3110ΔxylA::Cm^{r} (DE3). The final construct was transformed via electric shock using pET28a-cxylB and obtained a transformant, E. coli W3110ΔxylA::Cm^{r} (DE3)/pET28a-cxylB. The transformant was deposited into the Korean Collection for Type Cultures (KCTC) as KCTC 12100BP on Dec. 12, 2011.

<4-4> Preparation of E. coli BW25113ΔaldAΔxylA::Cm^{r} (DE3)/pET28a-cxylB

PCP20 plasmid was applied to E. coli BW25113ΔaldA::Kan^{r} and removed the kanamycin resistant gene. Then, xylA disruption cassette was applied to E. coli BW25113ΔaldA to prepare E. coli BW25113ΔaldAΔxylA::Cm^{r}. E. coli BW25113ΔaldAΔxylA::Cm^{r} prevented both b1 and b2 reactions (FIG. 2) by disrupting aldA gene as well as xylA gene in the biosynthesis route of the present invention. Verification of both genotypes and phenotypes of gene disruption was performed. Then, λDE3 prophage was inserted into E. coli BW25113ΔaldA::Cm^{r} using a λDE3 lysogenization kit (Novagen, USA), and finally obtained a construct of E. coli BW25113ΔaldA::Cm^{r} (DE3). The final construct was transformed via electric shock using pET28a-cxylB and obtained a transformant, The final construct was transformed via electric shock using pET28a-cxylB and obtained a transformant. The transformant was deposited into the Korean Collection for Type Cultures (KCTC) as KCTC 12117BP on Jan. 19, 2012.

<4-5> Biosynthesis of Ethane-1,2-Diol (Ethylene Glycol)

In order to perform the biosynthesis route of the present invention, ethane-1,2-diol was synthesized in E. coli prepared in Examples <4-3> and <4-4>.

First, 2 L of a fermentation medium containing 20 g of Bacto-tryptone, 10 g of Bacto yeast extract, 12 g of Na₂HPO4, 6 g of KH₂PO₄, 2 g of NH₄Cl, and 1 g of NaCl was prepared. Then, 80 g of a xylose solution and 0.48 g of MgSO₄ were respectively autoclaved and then added to the fermentation medium, while at the same time adding 80 μmol of kanamycin to the fermentation medium. An inoculum was prepared by introducing a single colony selected from an agar plate into a 5 mL LB medium containing chloramphenicol and kanamycin. The medium was cultured at 37° C. while stirring at a rate of 150 rpm. After 12 hours of culturing, the culture was transferred into 10 mL of a fresh LB medium containing chloramphenicol and kanamycin, and cultured for additional 12 hours. Then, the culture was transferred into a fermentation container and batch fermentation was started (t=0 h). The fermentation regulating conditions were set at 37° C., pH 7.0, with a stirring rate of 350 rpm, and under 0.5 vvm of air current. Then, concentrated NH₄OH and 3N H₂SO₄ were added thereto to maintain the pH of the culture, and 0.2 mL of 1 M isopropyl-β-D-1-thio galactopyranoside (IPTG) stock solution was added thereto, and the concentration of ethane-1,2-glycol was measured via GC and HPLC analyses.

<4-6> Measurement of Concentration of Ethane-1,2-Diol (Ethylene Glycol)

Extracellular metabolites such as xylose, xylonic acid, and ethane-1,2-diol were quantitated via HPLC analysis. More specifically, an HPLC analysis was performed in a Bio-Rad Aminex HPX-87H column (300×7.8 mm) at a flow rate of 0.4 mL/min using 0.5 mM H₂SO₄ as an eluent. The column was maintained at 55° C., and peaks were detected by Waters 2414 refractive index detector (FIG. 5).

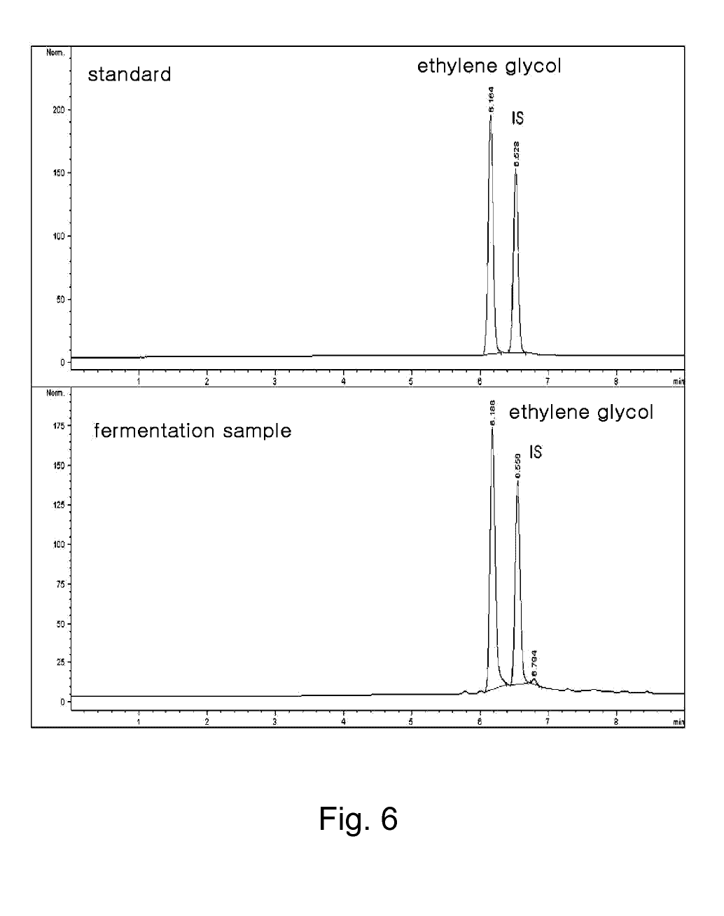

Additionally, the production of ethane-1,2-diol was quantitated via GC analysis and, more specifically, a GC (Agilent 6890N) equipped with a flame ionization detector (FID) and a HP-1 column (25 m×0.32 mm×0.17 μm). As a carrier gas, nitrogen gas with an inlet temperature of 200° C. and an uninterrupted flow rate of 14.10 mL/min was used. The oven program was set at 80° C. for 0.5 minute, increased up to 200° C. at a rate of 30° C./min, maintained thereat for 1 minute, finally increased up to 235° C. at a rate of 10° C./min, and then maintained thereat for 1 minute. FID temperature was set at 260° C., and 1,3-propanediol was used as an internal standard (FIG. 6).

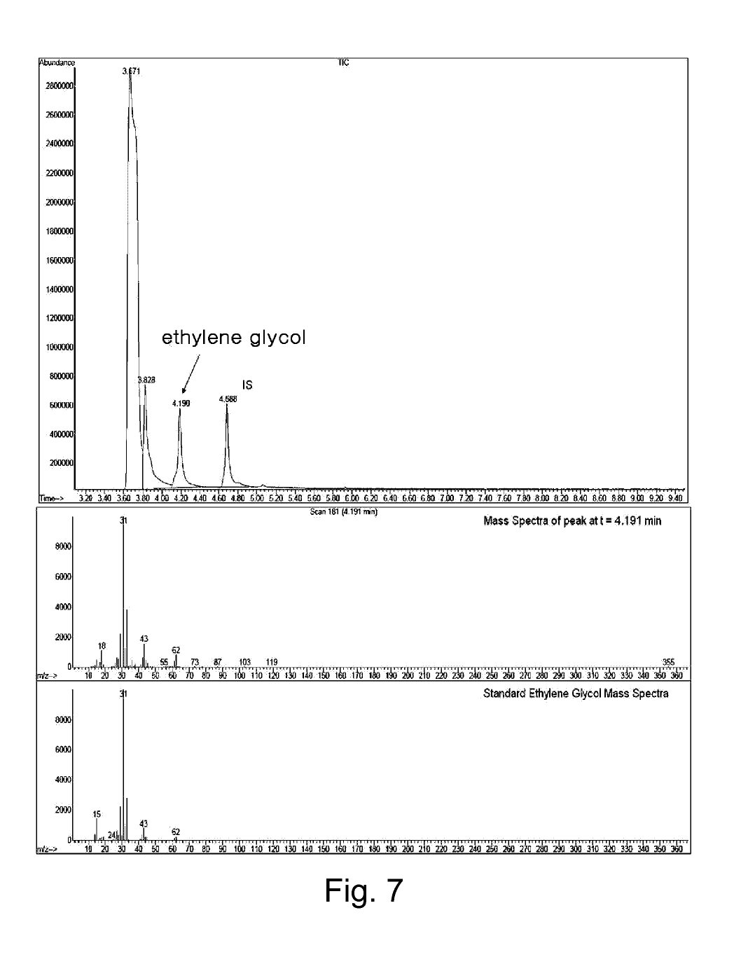

Additionally, the fermentation sample was analyzed via Gas Chromatography-Mass Spectrometry (GC-MS) and, more specifically, in a GC-MS (Agilent 6890, 5973MSD) equipped with a HP-5MS capillary column (60 m×0.25 mm×0.25 μm). Helium gas was used as a carrier gas. The oven program temperature and inlet temperature were set the same as in GC analysis, and 1,3-propanediol was used as an internal standard (FIG. 7).

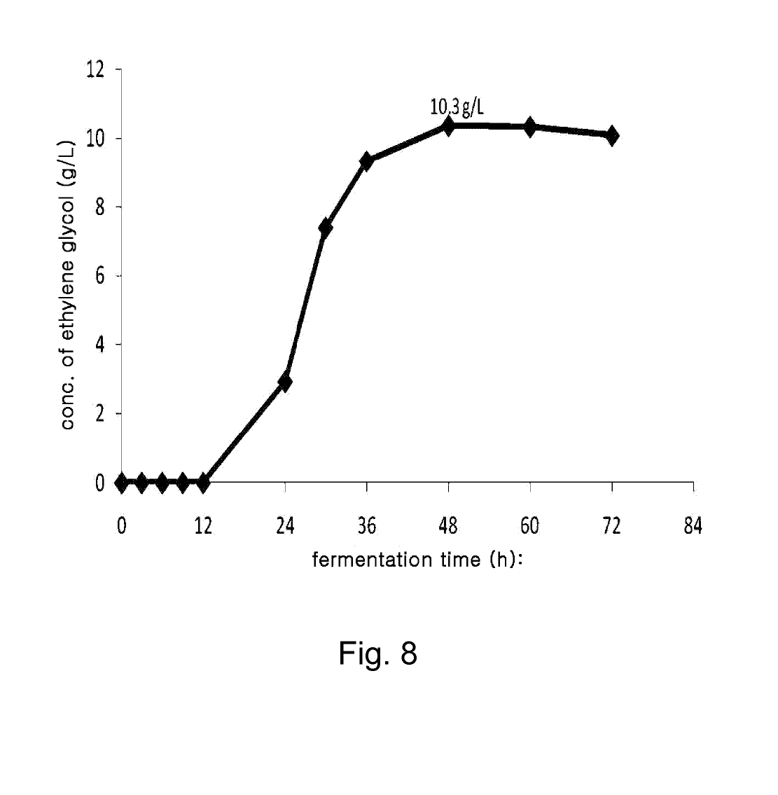

As a result, E. coli W3110ΔxylA::Cm^{r} (DE3)/pET28a-cxylB prepared in Example <4-3> successfully produced ethane-1,2-diol. More specifically, E. coli W3110ΔxylA::Cm^{r} (DE3)/pET28a-cxylB produced ethane-1,2-diol at a concentration of 10.3 g/L for 48 hours, representing an yield of 25.8% (FIG. 8). Additionally, acetic acid (0.5 g/L), formic acid (1.2 g/L) and ethanol (0.5 g/L) were produced at low concentrations 48 hours after the fermentation (Table 3).

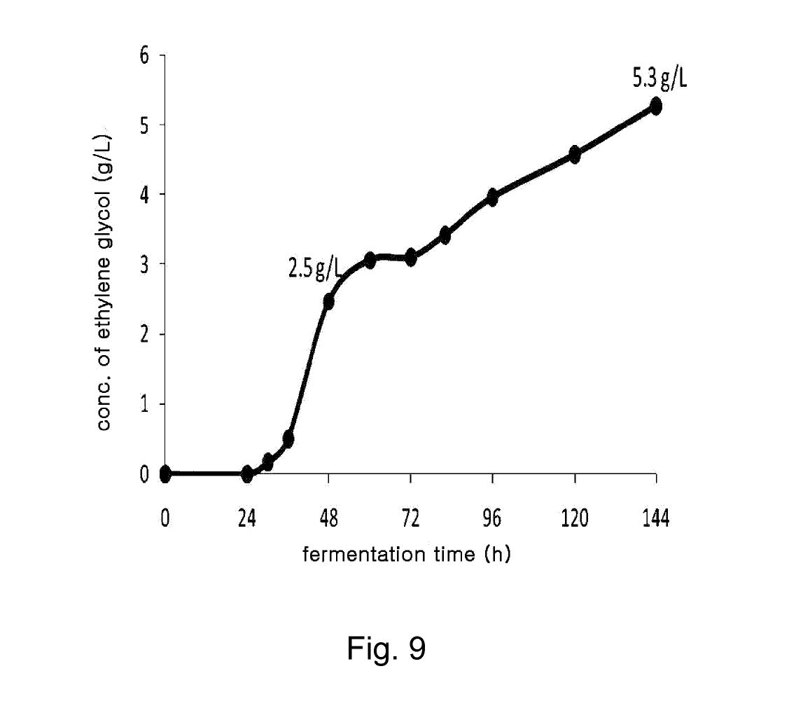

Meanwhile, E. coli BW25113ΔaldAΔxylA::Cm^{r}(DE3)/pET28a-cxylB prepared in Example <4-4> produced ethane-1,2-diol at a much lower level than that of E. coli W3110ΔxylA::Cm^{r} (DE3)/pET28a-cxylB. More specifically, E. coli BW25113ΔaldAΔxylA::Cm^{r}(DE3)/pET28a-cxylB produced ethane-1,2-diol at a concentration of only 2.5 g/L 48 hours after the fermentation, representing an yield of 6.3% (FIG. 9). The result confirmed by the analysis of metabolites that the disruption of caused a high accumulation of D-xylonic acid in a culture.

The accumulation of both D-xylonic acid and ethane-1,2-diol were increased to the extent of the extended fermentation time. However, the remaining D-xylonic acid in the culture was still high (16 g/L), even in the extended fermentation time (144 hours), and the concentration of ethane-1,2-diol reached only 5.3 g/L, representing an yield of 13.2% (Table 3).

TABLE 3
Concentration and yield of ethylene glycol produced
in E. coli using D-xylose

| | | Ethylene Glycol^{a} | |
|---|---|---|---|
| Entry | Strain | Conc. (g/L) | Yield (%) |
| 1 | E. coli W3110 (DE3), | n.d. | n.d. |
| | pET28a | | |
| 2 | E. coli W3110 | 10.3 | 25.8 |
| | ΔxylA::Cmr (DE3), | | |
| | pET28a-cxylB | | |
| 3 | E. coli BW25113 ΔaldA | 2.5 | 6.3 |
| | ΔxylA::Cmr (DE3), | | |
| | pET28a-cxylB | | |

^{a}Concentration 48 hours after fermentation; D-xylose (40 g/L) was depleted in all strains within48 hours after fermentation, and thus the yield was calculated based on the substrate (40 g/L);
n.d.: not detected).

### Example 5

### Designing of Additional Route for Optimizing the Biosynthesis of the Present Invention

The inventors of the present invention developed a method for improving the yield and concentration of products as a way to optimize the biosynthesis route for ethane-1,2-glycol production of the present invention. More specifically, in order to reduce carbon loss due to pyruvate formation (step 3 in FIG. 2), the inventors of the present invention designed two different routes for converting pyruvate into ethane-1,2-diol by employing two computer tools; i.e., PathComp (http://www.genome.jp) and ReBiT (Retro-Biosynthesis Tool, http://www.retro-biosynthesis.com) (FIG. 10).

Accordingly, it was confirmed that by combining the technology of converting pyruvate into ethane-1,2-diol, as described above, to the biosynthesis route for ethane-1,2-glycol production of the present invention, the efficiency of ethane-1,2-glycol production can be much improved.

### INDUSTRIAL APPLICABILITY

As described above, the biosynthesis of ethane-1,2-diol from a renewable biomass of the present invention provides a promising alternative to the conventional fossil-fuel-based method of producing ethane-1,2-diol, which has been generating global concerns on environment and instability due to the on-going depletion of fossil reserves; while also satisfying the continuously growing demand for ethane-1,2-diol.

Additionally, the biosynthesis method of the present invention using a microorganism does not require the high H₂ pressure and temperature for the hydrogenolysis of xylitol and thus enables efficient ethane-1,2-diol production.

Furthermore, the combination of the biosynthesis route of the present invention with a technology for pretreating plant supply materials will enable ethane-1,2-diol production in more cost-effective manner. Furthermore, large-scale production of ethane-1,2-diol will be possible by combining fermentation and metabolism engineering for the optimization of the biosynthesis route of the present invention.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. An engineered Escherichia coli (E. coli) capable of producing ethane-1,2-diol from D-xylose by knocking out D-xylose isomerase gene, xylA, within the genomic DNA of E. coli followed by transforming an expression vector including D-xylose dehydrogenase gene, cxylB, into the xylA-knockout E. coli.

2. The engineered E. coli of claim 1, wherein the E. coli was deposited into the Korean Collection for Type Cultures (KCTC) as KCTC 12100BP.

3. The engineered E. coli of claim 1, which is further capable of producing ethane-1,2-diol from D-xylose by knocking out aldehyde dehydrogenase gene, aldA within the genomic DNA of E. coli, wherein the transformed expression vector further includes the aldA-knockout E. coli.

4. The engineered E. coli of claim 3, wherein the E. coli was deposited into the Korean Collection for Type Cultures (KCTC) as KCTC 12117BP.

5. The engineered E. coli of claim 1, wherein D-xylose isomerase gene, xylA, includes a nucleotide sequence described in SEQ ID NO: 1.

6. The engineered E. coli of claim 3, wherein aldehyde dehydrogenase gene, aldA, includes a nucleotide sequence described in SEQ ID NO: 2.

7. The engineered E. coli of claim 1, wherein D-xylose dehydrogenase gene, cxylB, being derived from Caulobacter crescentus (C. crescentus), includes a nucleotide sequence described in SEQ ID NO: 3.

8. The engineered E. coli of claim 1, wherein the expression vector is pET28a vector.

9. The engineered E. coli of claim 1, wherein the E. coli strain is E. coli W3110 or E. coli BW25113.

10. A method for producing ethane-1,2-diol from D-xylose, comprising:
1) biosynthesizing ethane-1,2-diol by culturing the engineered E. coli of claim 1 in a medium containing D-xylose; and
2) obtaining ethane-1,2-diol from the cultured medium.

11. The method of claim 10, wherein, in step 1), the engineered E. coli is cultured in a fermenter via batch fermentation.

12. The method of claim 10, wherein the ethane-1,2-diol is biosynthesized in the engineered E. coli by a method comprising:
a) converting D-xylose into D-xylonic acid by D-xylose dehydrogenase;
b) converting D-xylonic acid into 2-dehydro-3-deoxy-D-pentonate by D-xylonic acid dehydratase;
c) converting 2-dehydro-3-deoxy-D-pentonate into glycoaldehyde by 2-dehydro-3-deoxy-D-pentonate aldolase; and
d) converting glycoaldehyde into ethane-1,2-diol by aldehyde dehydrogenase.

13. The method of claim 12, wherein, in order to convert pyruvate, a byproduct produced in converting 2-dehydro-3-deoxy-D-pentonate into glycoaldehyde in step c), into ethane-1,2-diol, the method further comprises:
e) converting pyruvate into acetyl-CoA by pyruvate dehydrogenase;
f) converting acetyl-CoA into citrate by citrate synthase by citrate synthase;
g) converting citrate into isocitrate by citrate hydro-lyase;
h) converting isocitrate into glyoxalate and succinate by isocitrate lyase;
i) converting glyoxalate into glycolate by glycolate oxidase;
j) converting glycolate into glycoaldehyde by aldehyde dehydrogenase; and
k) converting glycoaldehyde into ethane-1,2-diol by aldehyde dehydrogenase.

14. The method of claim 12, wherein, in order to convert pyruvate, a byproduct produced in converting 2-dehydro-3-deoxy-D-pentonate into glycoaldehyde in step c), into ethane-1,2-diol, the method further comprising:
l) converting pyruvate into phosphoenolpyruvate by phosphoenolpyruvate synthetase;
m) converting phosphoenolpyruvate into 2-phospho-D-glycerate by enolase;
n) converting 2-phospho-D-glycerate into glycerate by 2-phosphoglycerate phosphatase;
o) converting glycerate into hydroxypyruvate by hydroxypyruvate reductase;
p) converting hydroxypyruvate into glycoaldehyde and CO₂ by decarboxylase; and
q) converting glycoaldehyde into ethane-1,2-diol by aldehyde dehydrogenase.

15. A method of preparing an engineered E. coli capable of producing ethane-1,2-diol from D-xylose, comprising:
1) knocking out D-xylose isomerase gene, xylA, from a given E. coli;
2) constructing an expression vector including xylose dehydrogenase gene, cxylB; and
3) transforming the resulting expression vector in step 2) into the E. coli in step 1).

16. The method of claim 15, and further comprising knocking out aldehyde dehydrogenase gene, aldA, from the given E. coli.

17. The method of claim 15, wherein D-xylose isomerase gene, xylA, includes a nucleotide sequence described in SEQ ID NO: 1.

18. The method of claim 16, wherein aldehyde dehydrogenase gene, aldA, includes a nucleotide sequence described in SEQ ID NO: 2.

19. The method of claim 15, wherein D-xylose dehydrogenase gene, cxylB, being derived from C. crescentus, includes a nucleotide sequence described in SEQ ID NO: 3.
